(19) United States
(12) Patent Application Publication
(10) Pub. No.: US 2016/0338755 A1
(43) Pub. Date: Nov. 24, 2016

(54) NON-THERMAL PLASMA

(71) Applicant: LINDE AKTIENGESLLSCHAFT, Munich (DE)

(72) Inventors: Thomas Bickford Holbeche, Church Crookham (GB); Rodney Stewart Mason, Blakpill (GB)

(21) Appl. No.: 15/112,699

(22) PCT Filed: Jan. 22, 2015
(86) PCT No.: PCT/GB2015/000016

§ 371 Date: Jul. 20, 2016

(30) Foreign Application Priority Data
Jan. 23, 2014 (GB) 1401146.4

(51) Int. Cl. A61B 18/04; H05H 1/24

(52) U.S. Cl. CPC A61B 18/042; H05H 1/2406; A61B 2018/00744

(57) ABSTRACT

A plasma-generation device for applying plasma to a human body, having a reservoir containing a gas, a plasma zone in fluid connection with the reservoir, and means for generating a plasma by electrical discharge in the plasma zone. The gas has a composition of 92% to 99.9% Argon and 0.1% to 8% Krypton; or 95% to 99.5% Argon and 0.5% to 5% Hydrogen; or 92% to 99.5% Argon and 0.5% to 8% Nitrous Oxide.

The present disclosure relates to a non-thermal plasma treatment device and method. In particular, the disclosure relates to the production of so-called “cold plasma” and its application for treating various conditions. The treatments are preferably earned out in a medical or professional environment, or in the comfort of a user's home environment.

A gas is normally an electric insulator. However, when sufficient thermal energy is supplied to a gas or, alternatively, a sufficiently large potential difference is applied across a gap containing a gas, then it will break down and conduct electricity. This is because the electrically neutral atoms or molecules of the gas have been ionised to form electrons and positively charged ions. This ionised gas is a plasma.

When the ionisation is driven by a large potential difference, the momentum transfer between the light electrons and the heavier gas molecules and plasma ions is not very efficient. Therefore, the bulk of the energy that is supplied to form the plasma is supplied to the electrons. As a result, ionised gases, particularly at low gas pressures and charged particle densities, are described as “cold” or non-thermal. This means that the constituents e.g. the electrons, ions and gas molecules are each in thermal equilibrium only with similar mass species.

Such non-thermal plasmas are well known for use in destroying bacteria. For this reason, it is known to use non-thermal plasma in various forms of dental surgery. Due to the restrictions when operating in a patient's mouth, such plasma devices typically rely on a flow of gas between two electrodes to produce the plasma which can be directed onto the treatment area. The non-thermal production of the plasma provides a plasma gas having a temperature which is tolerable for the patient. WO2013040476 discloses a device for generating plasma which uses a flow of Helium.

Similarly, WO2012/042194 relates to the use of a plasma generator for the generation of plasma for oral treatments. The plasma generator generates a plasma using a Helium carrier gas with up to 40% of a more readily ionisable noble gas, selected from Argon, Krypton, Neon and Xenon.

It is also known to use plasma devices for treating skin infections. In such applications, the skin of the patient is typically used to provide the second electrode. In this way a first electrode can be held over the area to be treated and a large voltage difference is formed between the electrode and the patient's skin. This leads to the formation of a plasma from the gas between the electrode and the patient's skin. This allows for treatments of large areas through the use of a large electrode, but relies upon the formation of plasma from the air layer, U.S. Pat. No. 8,103,340, for example, uses such a device for treating a patient's skin.

It is known that the nature of the breakdown and the voltage at which this occurs varies with a wide number of parameters including the gas, the gas pressure, the materials and the nature, geometry and separation of the surfaces across which the potential difference is sustained, the separation distance of the electrodes and the nature of the high voltage supply.

It is known to use various gases when generating plasma for use in dental applications. It is typically known to use noble gases such as Helium or Argon. This is because these gases stabilise the plasma which is formed. These gases are not reactive but are excited to form a relatively long-lasting plasma and also serve to form reactive species with air, such as singlet Oxygen, hydroxyl radicals and the like.

DE102007040434 discloses a device for producing an electrical or electromagnetic field that is formed between a treatment probe and a body of a human or an animal. The probe is filled with a noble gas mixture of Argon and Neon.

WO2012172285 discloses a device for forming at an ambient atmospheric pressure a gaseous plasma comprising active species for treatment of a treatment region.

US2013233828 discloses an atmospheric plasma irradiation unit which has a discharge tube for ejecting a primary plasma formed of an inductively coupled plasma of an inert gas and a mixer for generating a secondary plasma formed of a mixed gas made into plasma by collisions of the primary plasma with a mixed gas region of a second inert gas and a reactive gas.

It is an object of the present invention to provide an improved approach to the use of plasma for various treatments, tackle the drawbacks associated with the prior art, or at least provide a commercially viable alternative thereto,

According to a first aspect, there is provided a plasma-generation device for applying plasma to a human body, the device comprising:
a reservoir containing a gas,
a plasma zone in fluid connection with the reservoir, and
means for generating a plasma by electrical discharge in the plasma zone,
wherein:
   the gas comprises from 92% to 99.9% Argon and from 0.1% to 8% Krypton; or
   the gas comprises from 95% to 99.5% Argon and from 0.5% to 5% Hydrogen; or
   the gas comprises from 92% to 99.5% Argon and from 0.5% to 8%) Nitrous Oxide.

The present disclosure will now be described further. In the following passages different aspects/embodiments of the disclosure are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

The present inventors have discovered that the efficacy of a plasma treatment can be enhanced by the doping of the basic plasma gas with a small amount of certain additional gases. These have been found to lead to an enhanced level of treatment, especially within the constraints of a hand-held treatment device,

The inventors have found that the inclusion of from 0.5 to 5% Hydrogen in Argon has a surprising efficacy in the treatments disclosed herein. More preferably the Hydrogen is present in an amount of from 1 to 2.5%, more preferably from 1 to 2%, and most preferably about 1.5%.

In particular, because Argon has a lower ionizing potential, it breaks down to form a plasma more readily and the increased rate of ionisation leads to a higher carrier gas temperature, compared to plasma gases such as Helium. This has rendered it unsuitable for use in most treatments applied to live cells or patients. Furthermore, the use of Argon as a plasma gas has been found to cause an unwanted breakdown of a stable glow discharge when excited by high voltage into a filamentary condition (including arcing). If this occurs in a plasma used in the bio-medical field it can lead to higher discharge currents being delivered to the subject than is acceptable.

It has surprisingly been found that the introduction of low levels of Hydrogen into the Argon helps mitigate these problems. Without wishing to be bound by theory, it is considered that the Hydrogen will help prevent the plasma temperature reaching unacceptable levels by partially quenching the plasma. It therefore tunes the rate of ionisation. As a related benefit, since it is a high thermal conductivity gas it additionally helps to carry heat away from the hot plasma regions. In contrast, pure Argon has a low thermal conductivity.

The inventors found that when the level of hydrogen was too low, the addition of hydrogen was insufficient to reduce the temperatures to a safe level. They also found that when the levels of the admixture of molecular Hydrogen to the Argon were too great, it would entirely quench the plasma produced. In the advantageous range discussed herein, it was found that the hydrogen would physically quench the very high energy states of Argon, but also produce atomic hydrogen in the plasma plume which, mixed with air at the site of application, affected the treatment site. It was theorised that the hydrogen may advantageously assist in the formation of hydroxyl radicals, since the presence of the hydrogen increased the efficacy of the treatment processes tested.

A particular advantage of using the Hydrogen and Argon blend is that Argon is a more cost-effective gas than Helium (a known alternative plasma gas). Accordingly, when part-quenched so that the plasma is at a useable temperature, the use of Argon is cheaper and at least as effective use of Helium as the plasma gas.

In addition, when the plasma plume is to be employed to supply an oxidative process for bleaching surfaces by formation of OH radical s, the extra supply of atomic Hydrogen available in the plume will allow more HO₂ and OH radicals to be formed when contacting molecular Oxygen available from the air and the NO also formed:

H+O₂->HO₂

HO₂+NO->OH+NO₂

The inventors have found that the inclusion of from 0.5 to 8% Nitrous Oxide in Argon has a surprising efficacy in the treatments disclosed herein. Preferably the gas comprises from 2% to 6% Nitrous Oxide. Without wishing to be bound by theory, it is considered that the Nitrous Oxide may allow more HO₂ and OH radicals to be formed when contacting molecular water and Oxygen available from the air.

The inventors have found that the inclusion of from 0.1 to 8% Krypton in Argon has a surprising efficacy in the treatments disclosed herein. More preferably the Krypton is present in an amount of from 0.5 to 6%, more preferably 1 to 5% and most preferably about 4%.

The presence of the Krypton in Argon was tested in experimental trials. It was found that this mix was the most efficient inert gas mixture in bleaching trials. Below 0.1% of Krypton the Argon causes sparks and arcing. Above 8% Krypton the plasma stops being formed because it is quickly quenched. In any event, the Krypton cost is high so it is desirable to avoid the use of too much Krypton gas.

It is believed that the presence of Nitrous Oxide or Krypton also have an effect on reducing the arcing associated with Argon being used as the plasma gas.

It is preferred that the above gases are supplied for use without the presence of any other gas species. That is, preferably the gas consists essentially of Argon and Krypton, Argon and Nitrous oxide, or Argon and Hydrogen, together with any unavoidable impurities. By unavoidable impurities, it is meant less than 5%, more preferably less than 1% and more preferably substantially no other gas species. It must be appreciated that, in use, the presence of air in the treatment zone will dilute the plasma gas which is employed.

The foregoing beneficial effects were demonstrated within the constraints of the device described below. As will be appreciated, such a device has restrictions on the practical gas pressure and flow rate that can be provided, as well as the electrical potential and treatment areas that can sensibly be employed,

The present invention relates to a plasma-generation device. That is, the device is designed to produce a plasma from the ionisation of a gas. The device is especially for producing a non-thermal plasma, as discussed herein. The plasma produced preferably has a temperature of less than 50° C., more preferably less than 48° C., more preferably less than 45° C. and most preferably from 37 to 42° C. It will be appreciated that for certain treatments, especially for hair treatment, temperature may suitably be at even higher temperatures. It is noted that the human pain threshold for temperature is typically around 48° C.

The device is suitable for applying plasma to a human body, which applies a number of constraints since thermal plasma production devices are clearly unsuitable. Furthermore, the production levels of UV, electrical stimulation and active species must be at levels which do not cause undue harm to a patient.

The device described herein is preferably hand-held. By hand-held, it is meant that at least the treatment application head is sized and configured such that it can be readily manipulated and controlled with one hand. Examples of hand-held devices include hair-brushes, hair-driers, foot-spa, hair-tongs, toothbrushes and the like. The treatment application head may be tethered to a power supply and/or a gas reservoir. Alternatively the treatment head may be fixed or pivotable with relation to an area to be treated. The device may also, for example, take a form such as a foot spa to allow ready treatment of an infected foot.

The ideal form for home use by a consumer is an entirely self-contained hand held device. This would have an internal battery as a power source and rely upon interchangeable gas canisters which can be clipped into the device. Nonetheless, for reasons of power requirements, it may be easier to have a mains power lead, attached to the device.

Especially when the device is to be used by a professional, such as in a hair or nail salon, or by a doctor, podiatrist, or the like, it may be easier to have the device tethered to a power supply and a larger gas tank. This makes it easier for the professional to use since they do not need to change the gas tank/cartridge/canister often.

Preferably the power supply comprises a battery integrated into the hand-held device. That is, preferably the plasma-generation device is entirely independent and does not require a tether to a power supply. This increases the utility of the device in-so-far as it can be more accurately applied and can be used in a wider range of environments, such as bathrooms,

The use of a device as discussed herein has a large number of advantages. The provision of the plasma keeps the device sterile and it can be readily reused for multiple patients, in addition, the plasma produces a ready supply of active gas species which provide the treatments discussed herein. The active gas species are further supplemented by the temperature, UV light and electrical stimulation which are associated with the plasma, production process.

The plasma treatment device comprises a reservoir containing the above-discussed gas. The reservoir acts as a source of gas from which a plasma is generated. The reservoir contains a source of pressurised gas which can be supplied to the plasma zone as the treatment application portion of the device. The gas will typically be stored in a tank (up to approximately 200 L) for professional use, or in replaceable and/or rechargeable canisters of cartridges for home use. The use, design and requirements for such sources of gas are well known in the art.

The reservoir is in fluid communication with a plasma zone within which plasma is created for treatment. In some embodiments the plasma zone is within the device and a flow of the plasma which is created leaves the device to provide the treatment. In other embodiments the plasma is formed directly at the site to be treated. The plasma zone includes means for generating a plasma by electrical discharge therein.

The device comprises a means for generating a plasma by electrical discharge through the gas. This can be achieved by one of several different approaches.

According to a first approach, the means for generating a plasma comprises a power supply and a dielectric electrode for placing in proximity to a human body, and wherein, in use, the plasma zone is formed between the dielectric electrode and a surface of a human body. The provision of a high voltage drop between the dielectric electrode and the human body leads to the production of a plasma between the dielectric electrode and the body, This is an effective way to treat a large area. The device of the present invention would preferably be configured such that the gases discussed herein can be flowed into the space formed between the dielectric electrode and the body, preferably at a relatively low flow rate, across substantially the whole area of the electrode.

According to a second approach the means for generating a plasma comprises a power supply, and first and second electrodes, and wherein, in use, the plasma zone is formed between the first and second electrodes and wherein a flow of gas from the reservoir through the plasma zone provides a flow of plasma to contact a surface of a human body. The provision of a high voltage drop between the two electrodes will cause the production of a plasma by ionising the gas provided. In this embodiment the gas flow will typically be greater so that the plasma flows out from between the electrodes and can be applied to a treatment area.

According to a third approach, the means for generating a plasma is a so-called surface micro discharge device. This comprises a power supply and first and second electrodes sandwiching a dielectric material. In use, a plasma zone is formed adjacent a surface electrode which can be held close to a surface of a human body. The provision of a high voltage drop between the electrodes leads to the production of a plasma across the area and, indeed, the electrode close to the treatment area will typically be a wire mesh. This is an effective way to treat a large area. The device of the present invention would preferably be configured such that the gases discussed herein can be flowed into the space formed between an external electrode on the device and the body, preferably at a relatively low flow rate, across substantially the whole area of the electrode.

Preferably the means for generating a plasma operates at a voltage of from 2-15 kV, preferably from 3 to 10 kV and most preferably about 5 kV. These levels of voltage can be achieved in a hand-held device and still produce a suitable level of plasma generation. The power range of the device is preferably 1-100 Watts AC at a high frequency of 10-60 KHz. Alternatively, power may be delivered as high frequency pulsed DC fast rise time square waveforms.

Preferably the gas is supplied through the means for generating a plasma at a flow rate of less than 51/min, preferably less than 2.51/min, more preferably less than l.51/min, preferably from 0. 1 to 11/min, preferably from 0.01 to 0.51/min. The gas flow rate for area treatments as discussed above will typically be lower than required for point treatments which require the production of a targeted jet of plasma. The flow rates for treatments which produce a plasma between a dielectric electrode a treatment are of a patient are preferably from 0.01 to 0.11/min. The flow rates for treatments which produce a plasma between two electrodes and rely on the gas flow to carry the plasma to a treatment are preferably from 0.5 to 2.51/min.

Preferably the device takes the form of a hair straightener, a toothbrush, a foot-spa or a hair-brush. In these recognisable forms, the consumer is already familiar with the usage requirements and application techniques required to employ the device. This avoids any hurdle to application. More particularly, these application devices are suitable for the application of the plasma to the regions that specifically require treatment, such as the hair or teeth of a user. The device may be a hand-piece for use by a podiatrist or a patient.

According to a further aspect there is provided a refillable canister for use in a plasma-generation device discussed above, especially a hand-held device. The canister will contain the gas blends discussed herein under pressure within a reservoir. Suitable pressures are from 1 to 200 Bar, more particularly from 20 to 100 Bar. During use the pressure of the stored gas blend will descend as the gas is used to form the plasma required for treatment.

According to a further aspect there is provided a kit comprising the device described herein and the refill canister described herein,

According to a further aspect there is provided a plasma for use in a method of treating a fungal infection in a nail, wherein the plasma is generated by electrical discharge through a gas, wherein
the gas comprises from 92% to 99.9% Argon and from 0.1% to 8% Krypton; or
the gas comprises from 95% to 99.5% Argon and from 0.5% to 5% Hydrogen; or
the gas comprises from 92% to 99.5% Argon and from 0.5% to 8% Nitrous Oxide.

This aspect of the present invention relates to the treatment of an infected nail. As will be appreciated, depending on the type of plasma generation device selected, the treatment may be applied to an entire nail or a portion of the nail. The treatment may be applied to an entire infected skin region or to only a portion. As a result, when treating only a portion of a nail or skin region it may be necessary to carry out a number of sequential plasma treatments. As discussed above, the plasma used in the present method is a cold or “non-thermal” plasma. This is essential when treating the human body since a thermal plasma would cause very severe tissue damage.

The inventors have found that the gas blends are particularly efficacious for the treatment of nails. In particular, the gases can be used to provide an efficacious topical application of non-thermal plasma to treat and prevent the spread of nail infections or onychomycosis caused by bacteria, fungi and other pathogens. The fungus discussed herein includes fungal species responsible, for example, for conditions such as athlete's foot. The use of the plasma treatment selves to ameliorate the infected toe and surrounding areas and to reduce the risk of the disease spreading or reoccurring,

The treatment especially relates to the treatment of human fingernails and toenails, and more particularly, to topical applications and methods to cure or prevent the spread of nail infections, such as onychomycosis, caused by bacteria, fungi and other pathogens. As will be appreciated, the treatment is for a fungal infection in and around the nail, but especially also under the nail where conventional treatments struggle to reach.

Onychomycosis is a nail disease of the toes and fingers typically caused by the organisms Candida albicans, Trichophyton mentagrophytes, Trichophyton rubrum, or Epidermpophyton floccusum. The nails become thickened and lustreless, and debris accumulates under the free edge. Nail plates becomes separated and the nails may be destroyed. It is acknowledged that the therapy of onychomycosis is difficult and protracted. Oral therapy with antimycotics requires months of administration and must be closely monitored for side effects.

Topical compositions have long been used with the objective of treating onychomycosis. Yet these chemical based topical applications have been largely unsuccessful because the nail is a difficult barrier for anti-fungal compounds to penetrate. To be effective a topical treatment for onychomycosis should exhibit a powerful potency for pathogens. It must also be permeable through the sail barrier, and safe for patient use. There exists a need in the art for a topical application that combines these traits in high degree. Moreover, there is a desire for a quick treatment time.

Non-thermal plasmas have long been known to exhibit biocidal properties yet none of the prior art has addressed the issue of targeting an infection under a nail and the associated permeability issues. Nor have they looked at the treatment of the pathogens that surround the infection, which is untreated, can lead to the spread of the infection or the re-infection of the digit.

Accordingly, there remains a need in the art for a topical application which can be safely applied to nails of fingers and toes, and which exhibits in combination permeability and potency for pathogens required to effectively cure, or prevent the spread of onychomycosis.

The compositions and method of the invention provide a unique means for treating onychomycosis. Advantageously, such means provides, in combination, certain characteristics, including safety, effectiveness, convenience, and freedom from toxicity, which have been unavailable heretofore. Through in vitro microbiological tests it is now found that a topical application of Plasma using the gas blends and device described herein, a topical application regime can be provided to a patient to effectively penetrate the nail and kill the bacteria causing the disease.

Without wishing to be bound by theory, the inventors speculate that the plasma treatment of an infected nail or skin region is driven by a number of mechanisms fuelled by the production of plasma-derived reactive Oxygen and nitrogen species (RONS). In particular, it is proposed that plasma treatment exerts its fungicidal action through the disruption of the cell exterior by increasing its permeability, resulting in a loss of membrane integrity and leakage of intracellular components. This cell death by necrosis may be mediated through more than one mechanism:
Production of transient pores by lipid and polysaccharide peroxidation induced by plasma-derived reactive Oxygen and nitrogen species (RONS);
Oxidation by RONS of protein thiol groups in the cell membrane and wall leading to their degradation;
Electroporation due to the residual electric current released from the device if the electric field exceeds ˜50 kV/cm,

It is hypothesized that the RONS generated from the interaction of ionised gas jet with air interact with water in nail, eventually creating OHONO (peroxynitrous acid). This molecule would act as an intermediate agent that permeates through the nail releasing OH which is most likely the final active species acting on the fungal cell.

Programmed cell death or apoptosis, another recognised cellular effect of plasma treatment in general, is believed to be a less relevant fungicide mode of action, except perhaps in the case of fungal spores. Apoptosis can occur when a compromised membrane structure (e.g. peroxidation) or change in membrane-bound proteins (e.g. ion channel proteins) activates intracellular signal pathways leading to complex cell responses ending in apoptosis. On the other hand, plasma-generated RONS themselves may penetrate into the cytoplasm inactivating the functional enzymes and other components within the cell, and inducing direct damage of DNA resulting in apoptosis.

UV radiation is likely to have a modest role in fungicidal action. Heat is not considered relevant in the efficacy of plasma as the induced surface temperature is below that resulting in thermal cell damage.

A preferred device for the treatment of nails is a foot-spa. Such a device would be designed to provide one or more plumes of plasma for treating a user's nails. The means for generating plasma would comprise first and second electrodes spaced around a plasma zone, and a flow of gas from the reservoir into the plasma zone to form plasma. The momentum of the gas which forms the plasma would direct the plasma onto the desired treatment area. Alternatively, the device may take the form of a single directable nozzle device.

According to a further aspect there is provided the use of a plasma for the cosmetic lightening of nails, wherein the plasma is generated by electrical discharge through a gas, wherein
the gas comprises from 92% to 99,9% Argon and from 0.1% to 8% Krypton; or
the gas comprises from 95% to 99.5% Argon and from 0.5% to 5% Hydrogen; or
the gas comprises from 92% to 99.5% Argon and from 0.5% to 8% Nitrous Oxide.

The inventors have discovered that the plasma produced using the above-discussed plasma gases further has an effect of bleaching a treated nail. It is typically the case that an infected nail will show some discolouration and will be yellowed. It is also known that the nails of smokers can become discoloured and even painted nails can retain some unwanted colouration when the paint is removed. The inventors have found that the gases are able to reduce the coloration of such nails so that they are lightened and a more natural colouration can be recovered. In particular, the inventors have found that the gases have an enhanced lightening effect, especially for a given treatment duration, compared to the use of Helium alone.

According to a further aspect there is provided the use of a plasma for the cosmetic whitening of teeth, wherein the plasma is generated by electrical discharge through a gas, wherein
the gas comprises from 92% to 99.9% Argon and from 0.1% to 8% Krypton; or
the gas comprises from 95% to 99.5% Argon and from 0.5% to 5% Hydrogen; or
the gas comprises from 92% to 99.5% Argon and from 0.5% to 8% Nitrous Oxide.

According to a further aspect there is provided a method for the cosmetic whitening of a tooth, the method comprising:
plasma treating a surface of a tooth with a plasma generated by electrical discharge through a gas, wherein
   the gas comprises from 92% to 99.9% Argon and from 0.1% to 8% Krypton; or
   the gas comprises from 95% to 99.5% Argon and from 0.5% to 5% Hydrogen; or
   the gas comprises from 92% to 99.5% Argon and from 0.5% to 8% Nitrous Oxide.

The inventors have found that within a fixed treatment time and especially under the conditions and limitations enforced by the use of a hand-held device, the use efficacy of the gas blends for whitening teeth was greater than that of Helium alone. In particular, for a given length of treatment time, the colour of the enamel was improved by a greater number of shades than under treatment of Helium alone, as in a conventional teeth whitening process.

The current process of cleaning teeth, involves a mechanical process of removing plaque (soft, sticky, bacteria infested film) and tartar (calculus) deposits that have built up on the teeth over time. This accumulation on the teeth provides the right conditions for bacteria to thrive next to the gums, which can lead to gum disease. By removing the plaque, you remove the bacteria's home, but much of the bacteria remain. The hope is that, deprived of a home, the body's normal defences and the active ingredients used in tooth paste will kill off the bacteria left behind and thus prevent, gum disease. However, this is often not the case.

In one aspect the invention seeks to provide a device for reducing the number of bacteria which survive a cleaning treatment, by providing a disinfection feature to the cleaning/prophylaxis process.

The inventors have found that it is possible to achieve this goal without necessarily having to add an additional tool or step. While bacteria populations may always recover, the Plasma discussed herein would significant reduce the bacteria load, particularly in the case of serious infections, thus significantly improving the chance that the body and twice daily brushing with be effective in preventing gum disease.

Accordingly, there is provided a device as discussed herein in the form of a toothbrush having an ultrasonic scaler head. In this way the provision of plasma is coupled with the ultrasonic scaling process. The integrated tool supports the prophylaxis process by simultaneously removing plaque and calculus while ‘washing’ the teeth and gum with plasma. The radicals contained in the plasma plume would kill and clean away the bacteria not removed with the plaque.

Preferably the ultrasonic scaler head would comprise a piezoelectric device to simultaneous provide the ultrasonics and to have incorporated therein one or a plurality of plasma gas outlets. Such a device would be small and convenient.

It is initially contemplated that such a device would comprise a hand-held portion and a base unit. The base unit would provide both the gas and a power supply. The head would simply incorporate a small transformer and electrode. In this way the hand-held device would not be unwieldy for its intended purpose.

Preferably the transformer could be wound coaxially to the plasma chamber to help keep overall diameter within accepted hand piece range. The plasma chamber could include a self-closing valve arrangement that allows sealing from the autoclave and helps prevent contamination of the chamber. The end of the hand piece would preferably engage the standard ISO fitting and align with the gas delivery channels and electrical contacts therein. The high voltage generating parts would be contained in the removable hand piece section and potted with a suitable resin/silicone compound that resists autoclave temperature and moisture ingress,

Such a device represents a significant improvement in the process, as it would significantly increase the ‘anti-bacterial’ effect of the prophylaxis process. Therefore greatly reducing the risk of gum disease. Where a dentist or hygienist applies an active disinfectant to the mouth, it would eliminate this step and any risk associated with using chemicals in the mouth. The device could be extended, to treat the bacteria deep in the gum pockets. The device described above will provide some disinfection of pockets, yet to get deeper into these areas, a specific pocket probe could be included

The plasma could also generate water spray, thus eliminate the need for the ultrasound to atomise the water. This may have the added benefit of making the water ‘active’ to enhance the anti-bacterial properties.

According to a further aspect there is provided the use of a plasma for the cosmetic bleaching of hair, wherein the plasma is generated by electrical discharge through a gas, wherein
the gas comprises from 92% to 99,9% Argon and from 0,1% to 8% Krypton; or
the gas comprises from 95% to 99.5% Argon and from 0.5% to 5% Hydrogen; or
the gas comprises from 92% to 99.5% Argon and from 0.5% to 8% Nitrous Oxide.

According to a further aspect there is provided a method for the cosmetic bleaching of a hair, the method comprising:
plasma treating a surface of a hair with a plasma generated by electrical discharge through a gas, wherein
   the gas comprises from 92% to 99.9% Argon and from 0.1% to 8% Krypton; or
   the gas comprises from 95% to 99.5% Argon and from 0.5% to 5% Hydrogen; or
   the gas comprises from 92% to 99.5% Argon and from 0.5% to 8% Nitrous Oxide.

As with the lightening of nails and the bleaching of teeth, the inventors have found that within a fixed treatment time and especially under the conditions and limitations enforced by the use of a hand-held device, the use efficacy of the gas blends for bleaching hair was greater than that of Helium alone. In particular, for a given length of treatment time, the hair was lightened more than under treatment of Helium alone.

According to a further aspect there is provided a method for the cosmetic dyeing of hair, the method comprising:
plasma treating a surface of a hair with a plasma generated by electrical discharge through a gas, wherein
   the gas comprises from 92% to 99.9% Argon and from 0.1% to 8% Krypton; or
   the gas comprises from 95% to 99.5% Argon and from 0.5% to 5% Hydrogen; or
   the gas comprises from 92% to 99.5% Argon and from 0.5% to 8% Nitrous Oxide; and
applying a hair-dye to the plasma-treated hair.

The inventors have found that the use of a plasma pre-treatment, especially with the gases discussed herein, leads to an improved longevity of hair dye. In particular, the use of a typical 3 wash dye could be extended to match an equivalent semi-permanent hair dye. Similarly, a typical 28 wash dye could be extended to match a 40 wash permanent dye. This is especially advantageous because the harsh chemicals required for a longer lasting hair dyeing process can be avoided and less damaging chemicals can be used to achieve the same long lasting colour.

As should be appreciated, all of the gas blends discussed herein are suitable for use in each of the foregoing aspects, treatments and uses. Accordingly, all of the preferred features relating to the gas blends apply equally to each of the aspects.

As should further be appreciated, the foregoing uses, methods and treatments are all suitably performed using the device as discussed herein. In particular, the device can be readily adapted for use in each of the foregoing uses, methods and treatments to ensure that a suitable amount of plasma is provided at a target location to thereby put the invention into effect.

In combination with the foregoing methods, when treating a nail with the plasma gas discussed herein, it is possible and may be desirable to pre-treat the nail surface. This may be performed by abrading the surface with a nail file or by drilling holes, grooves or channels into the surface of the nail. This reduces the thickness of the nail to be treated and can help active species penetrate into the nail and come closer to the nail-bed. An example of such a pre-treatment is provided by controlled micro penetration (CMP) by Clearanail™. When drilling holes in the nail it is desirable that the full thickness of the nail is not penetrated to avoid the risk of infection or damage to the nail bed. Typical holes may be drilled 2-3 mm apart. The holes or grooves are spaced to prevent significant reduction in the structural rigidity of the nail. Pre-treatment increases the efficacy of the treatment. Preferably after treatment with plasma the nail may be coated in a lacquer to prevent infection through the thinned portions and to provide support to the nail integrity.

### FIGURES

The present disclosure will be described in relation to the following non-limiting figures, in which:

FIG. 1 depicts a device for the production of a plasma gas flow. The device is shown in full in FIG. 1A and in close cross-section in FIG. 1B. The device has an inner conductor and an outer conductive sheath sandwiching an inner conductor having gas channels for the passage of the plasma gas blend.

FIGS. 2A and 2B depict a device for the production of a plasma gas flow. The device includes a single electrode having through-holes for the passage of gas provided by and underlying tortuous gas conduit. There may further be provided a heater below the gas conduit to heat the whole assembly. Such a configuration would be suitable for hair-straighteners.

FIG. 3 shows an exemplary hair straightening tool employing the plasma device plates shown in FIG. 2.

FIG. 4 shows a schematic of the components which may be required for establishing a plasma flow for treatment.

FIG. 5 shows various views of a PF4 test rig as described herein. The rig includes a hand-held applicator 500 tethered to a gas supply 501 within the body of the rig. The body of the rig contains certain of the control electrics.

FIG. 6 shows two close-up view of the hand-held applicator 500 shown in FIG. 5. This shows the configuration of the device including the gas flow pathway from the gas supply 501 to the nozzle via a valve and between a pair of electrodes for the generation of the plasma.

Preferred embodiments of devices that apply the principles of the invention set out above will now be described with reference to the accompanying Figures.

Plasma application devices 100, 300, 400 may comprises: a source of gas in communication with one or more gas outlets 125, 325, 425, and a first electrode 110, 310, 410. Optionally, a second electrode 130, 330, 430 may also be provided. Alternatively, the second electrode may be formed by the article to which the plasma is to be applied (in which case it is not considered to form part of the device).

The source of gas may be a gas reservoir enclosed within the plasma application device, or may be a conduit in communication with a separate gas supply.

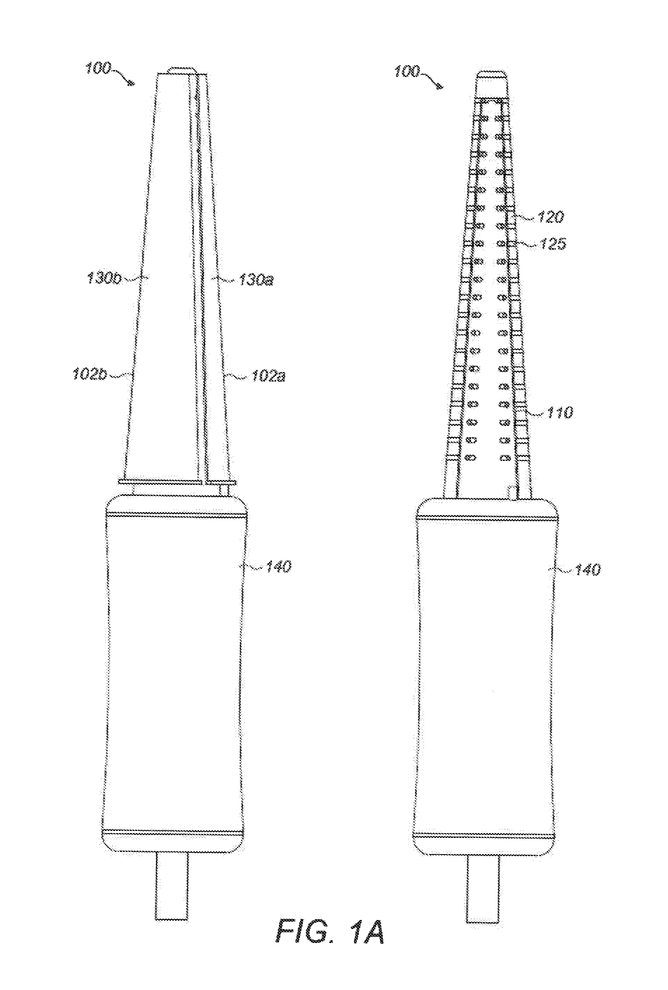

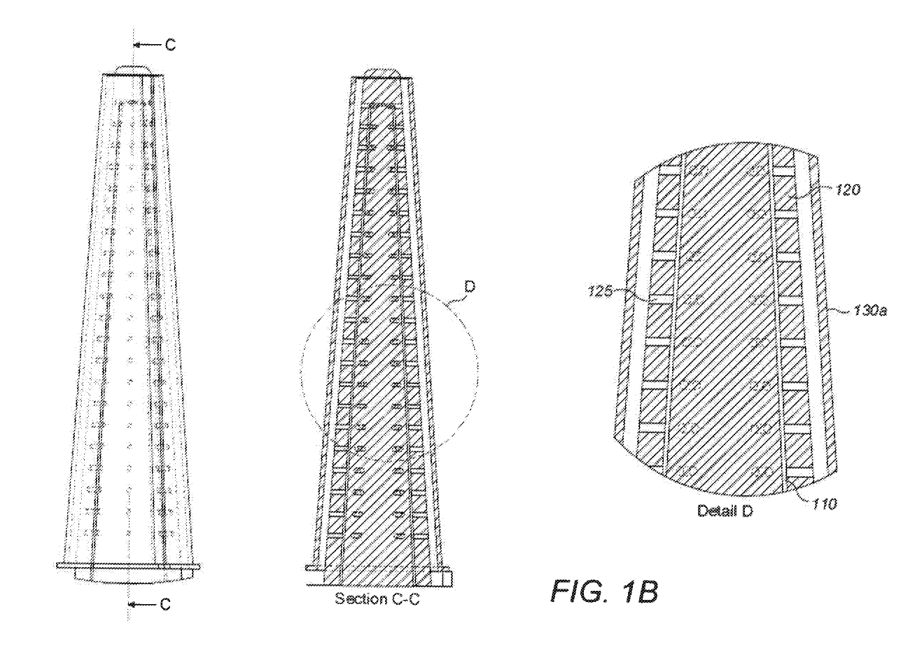

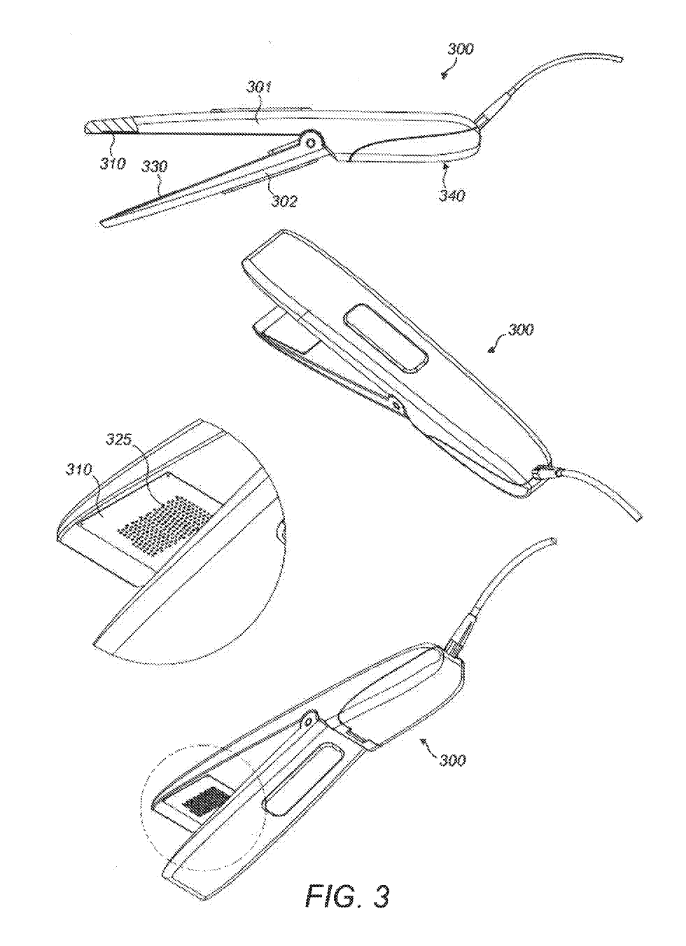

FIGS. 1 and 3 show plasma application devices 100, 300 for applying plasma to an article in which the device comprises a second electrode 130, 330. The article is to be located between the first electrode 110, 310 and the second electrode 130, 330. For this purpose, the second electrode 130, 330 may be movable relative to the first electrode 110, 310.

In the embodiment of FIG. 1, which may form a device for curling hair, at least two second electrodes 130a, 130b are provided, such that an electric field may be established between the first electrode 110 and either (or both) of the second electrode(s) 130a, 130b.

Preferably, the at least two second electrodes 130a, 130b substantially surround the first electrode 110. The at least two second electrodes 130a, 130b may comprise or be formed of a conductive polymer.

A housing 120 may surround the first electrode 110, with the one or more gas outlets 125 formed in the housing 120. Such a housing may comprise or be formed from a dielectric material such as a ceramic. Alternatively to the arrangement of FIG. 1, the housing 120 may itself form the first electrode 110 with the one or more gas outlets 125 forming a through-hole in the first electrode 110.

Preferably, a plurality of gas outlets 125 are provided spaced over a portion of the surface of the housing 120. Preferably, the gas outlets 125 are arranged such that gas passing through the gas outlets 125 will contact the second electrode 130.

The at least two second electrodes 130a, 130b may substantially surround the housing 120 so as to align with the plurality of gas outlets 125. The at least two second electrodes 130a, 130b may be movable (for example, pivotable) relative to the housing 120 for clamping an article (for example, hair) therebetween. A switch or sensor may be provided to trigger the device 100 to provide plasma when the second electrodes 130a, 130b are in a predetermined position relative to the first electrode 310.

The housing 120 may be generally cylindrical or generally conical or frusto-conical in shape. The at least two second electrodes 130a, 130b may be complementary in shape with the housing 120.

The device 100 may comprise a handle 140. The source of gas may be a reservoir located within the handle 140.

In use, the article may be passed between the housing 120 and the at least two second electrodes 130a, 130b. A plasma may be applied to the article by passing a gas from the source of gas via the one or more gas outlets 125 to the article at a location between the first electrode 110 and the second electrode 130. A voltage is applied between the first and second electrodes 110, 130 thus ionises the gas to form the plasma. Preferably, the second electrode 130 is connected to earth, while high frequency signal is applied to the first electrode 110.

In the embodiment of FIG. 3, which may form a device for straightening hair, the first electrode 330 may be mounted on or form a first component of a housing of the device 301 while the second electrode 330 may be mounted on or form a second component of a housing of the device 302. The first and second components of the housing 301, 302 may be pivotably connected, thereby allowing relative movement between the first and second electrodes 110, 310. Such movement may allow the user of the device to clamping an article (for example, hair) therebetween. A switch or sensor may be provided to trigger the device 100 to provide plasma when the second electrode 330 is in a predetermined position relative to the first electrode 310.

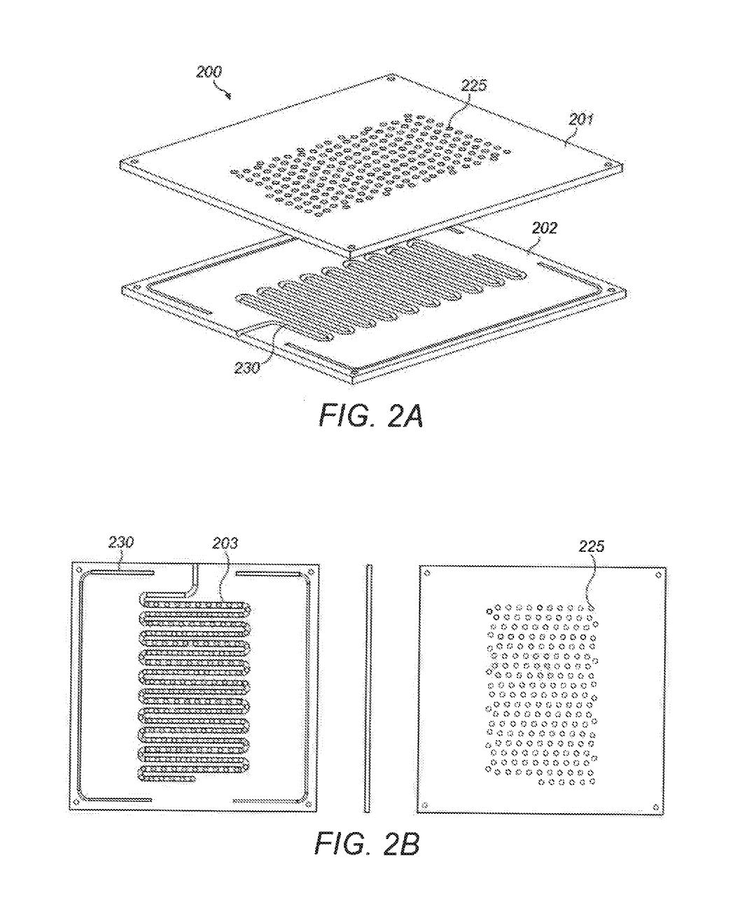

Preferably, the at least one gas outlet 325 is formed as a through-hole penetrating the first electrode 310. A suitable example of such an electrode is shown in FIG. 2A and described in detail below.

The device 300 may comprise a handle 340. The source of gas may be a reservoir located within the handle 340.

FIG. 2 depicts an electrode 200 for the production of a plasma gas flow. The electrode comprises a plurality of through-holes 225 for the passage of gas.

The electrode 200 may be formed a first conductive plates 201 and a second conductive plate 202. In use, the first plate 201 forms the article facing surface of the electrode. The plates 201, 202 may comprise a ceramic such as aluminium nitride.

The through-holes 225 may be formed in the first plate 201. A groove 230 may be formed in the second plate 202. The groove 230 is arranged to coincide with the through-holes 225. The first plate 201 may be affixed to the second plate 202 (for example, using fasteners or adhesive). The groove 230 extends from an edge of the second plate 202, at which edge it forms a gas inlet 203 for the electrode 200. Preferably, the groove 230 forms a single continuous conduit between the first and second plates 201, 202.

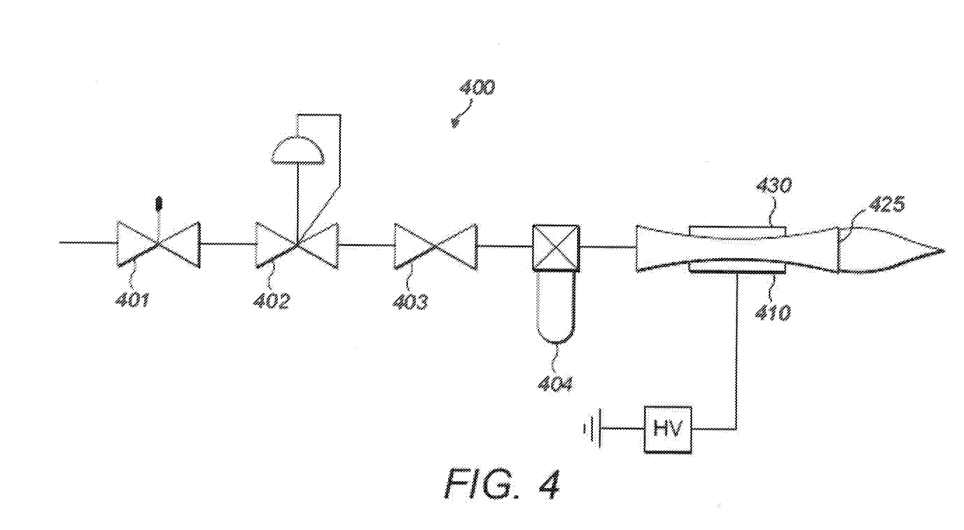

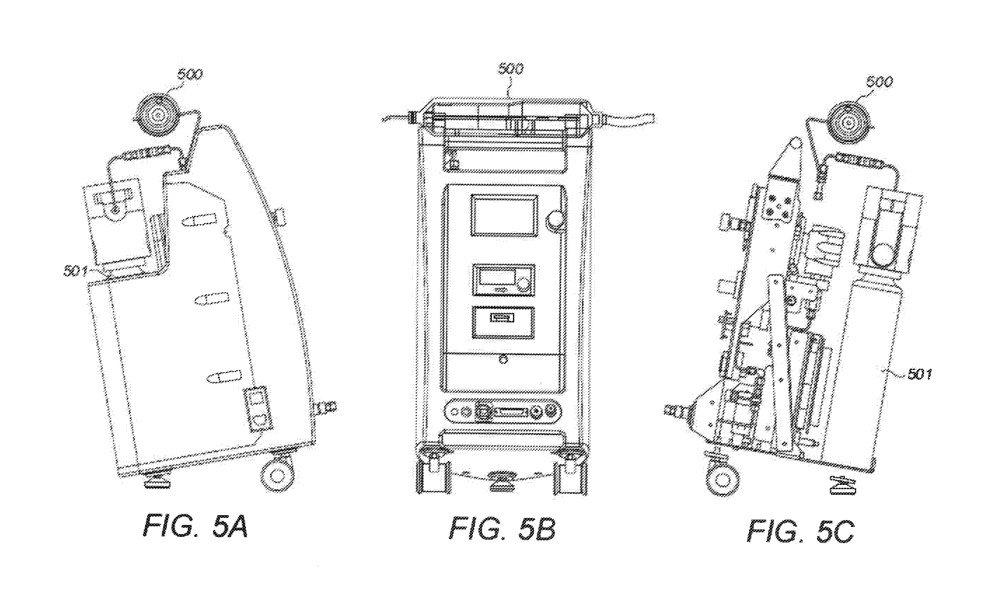

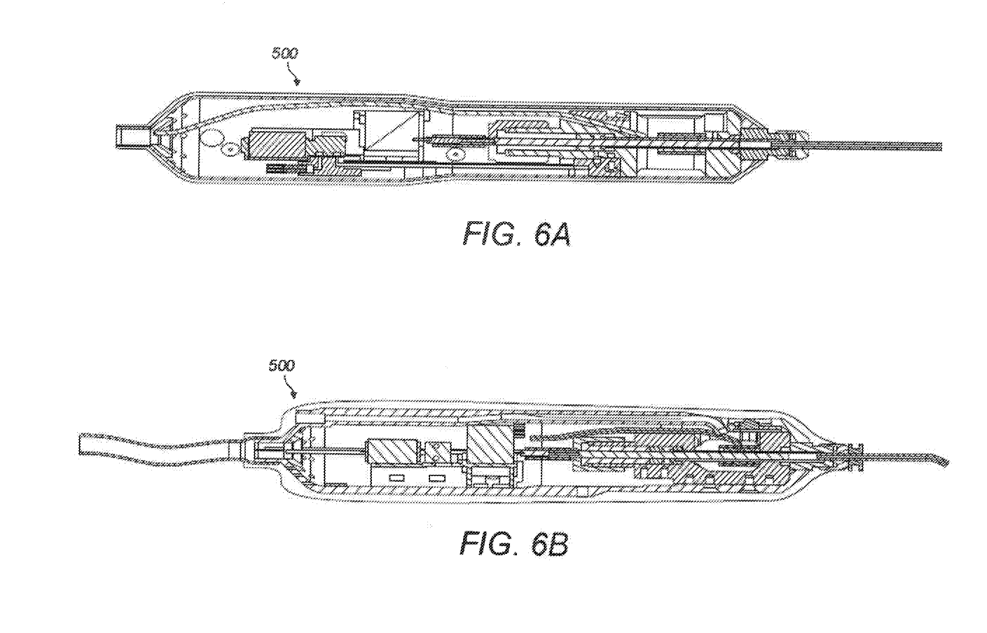

Optionally, there may be provided a heat source below the second plate 202, (for example, below the conduit) to heat the electrode 200. The use of a heater lowers the energy required for the gas to form a plasma.

Whereas the specific embodiments depicted in FIGS. 1 and 3 are preferably for applying plasma to an article located between two electrodes, the invention as described above can be applied to create a jet of plasma. FIG. 4 shows an example of such a plasma application device 400. The device may be used to apply plasma to a surface of an article, such as a hand or region of skin.

Plasma application device 400 comprises a source of gas. The source of gas may comprise in series: a needle valve 401; a pressure regulator 402; a mass flow meter 403; and a sintered element 404.

The source of gas is arranged to provide a flow of gas between two electrodes 410, 430. Whilst the electrodes 410, 430 are depicted as being separated such that the flow direction is perpendicular to their separation, this is not essential In fact, the electrodes 410, 430 may be separated in the direction of the gas flow. The gas may be ejected from the device 400 via one or more gas outlets 425. The one or more gas outlets 425 may be located downstream of the electrodes 410, 430. A nozzle may be provided downstream of the electrodes 410, 430. Alternatively, one of the electrodes 410, 430 may form the nozzle.

In an alternative embodiment, only a single electrode 410 is provided with the article acting as the second electrode. Thus, the source of gas is arranged to provide a flow of gas past the single electrode 410. The gas may be ejected from the device 400 via one or more gas outlets 425. The one or more gas outlets 425 may be located downstream of the electrodes 410, or may be formed as through holes in the electrode 410 (for example, in the manner depicted in FIG. 2. A nozzle may be provided downstream of the electrode 410. Alternatively, the electrode 410 may form the nozzle.

### EXAMPLES

The present disclosure will now be described in relation to the following non-limiting examples.

There are many possible uses for cold atmospheric plasmas. The aim of these trials was is to analyse the bleaching efficacy of a variety of gas mixtures at different concentrations, whilst measuring the levels of ozone and nitrous oxide produced, recording the voltage deposition on a “wet human” test model and determining the temperature of the plume. Optical spectra were also taken in order to analyse the levels of certain rnetastable states and excited radicals.

Part A—Comparative Testing of Gas Mixes Using ParaSure Plasma Indicator Strips

The objective was to find the most efficient plasma gas mix within necessary safety limits for a commercially viable device. This was done by assessing the bleaching efficacy of a variety of gas mixtures at different concentrations whilst also measuring the undesirable by-products of ozone and NOx and the temperature and electrical leakage down the plume,

The following tests were carried out using an experimental rig with the internal reference PF4. This includes a base control unit provides the required gas flow and electrical supply via an umbilical cord to a hand held unit. The hand held unit consists of concentric inner and outer barrier electrodes mounted on quartz tubes to which a high voltage is applied and between which the gas is flowed. The discharge plasma gas flows down the open quartz flow tube and in to the atmosphere. The main discharge strikes across the narrow gas between the inner and outer electrodes but a secondary discharge occurs down the flow tube in to the plume formed by the flow of plasma gas mixing with the air at the end of the flow tube.

The gas flow rate used was 1.5 L/m. The power settings were varied to create different levels of plasma excitation and the gas mixes were varied by means of two mass flow controllers. The L*a*b* colour of the strips was measured using a spectrophotometer and the rate of bleach standardised to a measure of time to achieve a change of 2.5 or 5% L*SCI.

The best results per gas mix/power setting combination are presented.

Test A1 - Helium based mixes

| | | | Bleach | | | | |
|---|---|---|---|---|---|---|---|
| Test | | | speed | Temperature | Ozone | | |
| sample | Gas used | Voltage kV | (mins) | (deg C.) | (ppb) | NOx (ppb) | Comments |
| 1 | He (control) | 7 | >120 | 38 | 35 | 25 | Very slow |
| 2 | He | 7 | 75 | 28 | 21 | 21 | Quicker if |
| | 1% Ar | | | | | | pulsed |
| 3 | He | 7 | >120 | 28 | 27 | 20 | |
| | 4% Ar | | | | | | |
| 4 | He | 7 | 65 | 29 | 41 | 340 | |
| | 8% Ar | | | | | | |
| | He 1% Kr | 7 | 16 | 40 | 50 | 20 | |
| 5 | He | 7 | 9 | 30 | 25 | 210 | Fast |
| | 2% Ne | | | | | | |
| 6 | He | 7 | 9 | 30 | 4 | 130 | Fast |
| | 8% Ne | | | | | | |
| 7 | He | 7 | 18 | 28 | 5 | 85 | |
| | 20% Ne | | | | | | |
| | He (control) | 9 | 20 | 42 | 45 | 120 | |
| 8 | He | 9 | 8 | 40 | 80 | 20 | Fast |
| | 1% Kr | | | | | | |
| 9 | He | 9 | >120 | 44 | 65 | 16 | |
| | 2% Kr | | | | | | |
| 10 | He | 9 | >120 | 35 | 4 | 120 | |
| | 1% H2 | | | | | | |
| 11 | He | 9 | >120 | 36 | 5 | <5 | |
| | 2% H2 | | | | | | |
| 12 | He | 9 | >60 | 34 | 35 | — | |
| | 10% Xe | | | | | | |
| 13 | He | 9 | >60 | 35 | 18 | — | |
| | 20% Xe | | | | | | |
| 14 | He | 9 | 14 | 35 | 17 | 360 | |
| | 1% N2 | | | | | | |
| 15 | He | 7 | <5 | — | — | — | |
| | 250 ppm O2 | | | | | | |
| 16 | He | 7 | <5 | — | — | — | |
| | 500 ppm O2 | | | | | | |
| 17 | He | 7 | 5 | — | — | — | High NOx |
| | 2% N2O | | | | | | |

The data show that:
Various additions of inert gases to He can significantly improve the oxidative effectiveness of the plasma beyond that possible with He alone.
Different gas mixes produce significantly different oxidation results.
Different concentrations of a gas mix produce different results and it can be seen that different concentrations work better for different gases,
Kr, O₂ and Ne are the most effective additions with the Ne mix being less sensitive to concentration.

Test A2 - Argon based mixes

| | | | | Tem- | | | |
|---|---|---|---|---|---|---|---|
| Test | | Volt- | Bleach | pera- | | | |
| sam- | Gas | age | speed | ture | Ozone | NOx | |
| ple | used | kV | (mins) | (° C.) | (ppb) | (ppb) | Comments |
| 1 | Ar | 5-9 | n/a | >150 | n/a | n/a | Arcs and |
| | (control) | | | | | | very hot |
| 2 | Ar | 7 | 7 | 34 | 27 | 59 | Fast |
| | 1% Kr | | | | | | |
| 3 | Ar | 7 | 2 | 39 | 13 | 100 | Very Fast |
| | 4% Kr | | | | | | |
| | Ar | 7 | — | — | — | — | Arcing |
| | 8% Kr | | | | | | |
| 4 | Ar | 9 | 31 | 32 | 4 | 120 | |
| | 1% H2 | | | | | | |
| 5 | Ar | 9 | 12 | 30 | 6 | 160 | |
| | 2% H2 | | | | | | |
| 6 | Ar | 9 | 29 | 30 | 9 | 105 | |
| | 4% H2 | | | | | | |
| 7 | Ar | 9 | 20 | 31 | 44 | 1410 | NOx very |
| | 1% N2 | | | | | | high |
| 8 | Ar | 9 | 26 | 29 | 20 | 1290 | NOx very |
| | 2% N2 | | | | | | high |
| 9 | Ar | 9 | >26 | — | — | 2200 | NOx very |
| | 4% N2 | | | | | | high |
| 10 | Ar | 9 | 1 | — | — | — | NOx very |
| | 2.4% | | | | | | high |
| | N2O | | | | | | |

The data show that:
Pure Ar arcs easily and would require undesirably high gas flow rates to control,
At lower, economically acceptable and practical flow rates Pure Ar benefits from a molecular gas to quench its tendency to arc rather than form a stable plasma. The addition of N₂ or N₂O produced unacceptable levels of NOx. The most effective molecular gas mixes were therefore the Ar/H2 mixes,
The addition of Kr to Ar produces the most efficient and effective result within acceptable safety limits.

Part B—Comparative Testing of Gas Mixes Using Saccharomyces cerevisiae as a model for Trichophyton Rubrum

The objective was to find whether any of the gas mixes from Part A could exhibit a biocidal effect against a cultured yeast under a variety of conditions.

The following tests were carried out using a plasma test device with gas flow rates of 1.5 L/min.

Test B1—Qualitative Assessment of Direct Exposure to Agar Plates

A suspension of S. cerevisiae was prepared by adding colonies from an agar plate to 3ml of PBS. This was prepared to an optical density of 0.2 measured using the spectrophotometer with PBS only as a blank.

To obtain an even growth of S. cerevisiae on the surface of the Malt Extract Agar, 200 ul of the 0.2 OD suspension was pipetted on to the agar. This was spread evenly around the plate's surface using a plastic spreader.

The plasma plume was aimed at the centre of the inoculated agar plates for the specified durations and qualitative observations of the fungicidal effect were made following 48 hr incubation.

| Test | | | |
|---|---|---|---|
| # | Description | He/1% Ar | Ar/4% Kr |
| 1 | 10 seconds | Very small zone | Small zone of |
| | | of reduced growth | inhibition |
| 2 | 30 seconds | Small zone of | Medium zone of |
| | | reduced growth | inhibition |
| 3 | 2 minutes | Medium zone of | Large zone of |
| | | inhibition | inhibition |
| 4 | Control - no | No inhibition | No inhibition |
| | treatment | | |
| 5 | Control - Gas | No inhibition | No inhibition |
| | only 2 mins | | |

The data show that:
The gas mix plasmas did exhibit a zone of inhibition proportional to the duration of application.
The 4%Kr/Ar mix produced a larger zone of inhibition than the 1%Ar/He mix in the same time.

The gas only control produced no zone of inhibition.

Test B2—Quantitative Assessment of Direct Exposure to Broth Cultures

Colonies from a plate containing S. cerevisiae were picked off and added to 10 ml of malt extract broth containing ceftazidime to create a broth culture. Microtitre wells containing 30 uL of 1.0 OD concentrated broth incubated for 48 hours were then exposed to plasma for differing time periods. The wells were then rehydrated with PBS, serially diluted and plated out to obtain cell counts.

Cell counts made before and after plasma treatment from average of 9 individual wells at 10⁻¹ dilution.

| Test | | | |
|---|---|---|---|
| # | Description | He/1% Ar | Ar/4% Kr |
| | Initial broth | >500 | >500 |
| 1 | Control - gas only | 64 | 23 |
| | 5 mins | | |
| | Control - no | 34 | 34 |
| | treatment | | |
| 2 | 2 mins plasma | 29 | 0 |
| 3 | 5 mins plasma | 0 | 0 |

The data show that:
The Ar/4%Kr mix reduced the colony count to zero more quickly than the He/1%Ar mix was able to and is therefore confirmation of its superior fungicidal properties.

Test B3—Quantitative Assessment of Exposure to Broth Cultures Through Nail

40 uL of the same broth culture used above was added to a modified Franz Cell within which a human nail clipping was secured. The Franz cell was inverted to allow the broth to remain in contact with the underside of the nail and the plasma applied for varying durations to the nail surface. The cell was then incubated and washed out using 100 uL of PBS, serially diluted and plated out for colony counting.

The seal around the edge of the nail meant that any measured reduction in the colony count in the broth would have to be as a result of plasma acting through the nail.

This test was done by applying plasma for 15 minutes using just the He/1%Ar mix in order to assess nail penetration. Each data point is the average cell count of 3 replicates.

Cell counts were taken before and after plasma treatment at different broth culture starting concentrations.

| Test | | | |
|---|---|---|---|
| # | Description | Neat | 10⁻¹ |
| 1 | Nail 0.4 mm thick | 42 | 4 |
| 2 | Nail 0.7 mm thick | 30 | 4 |
| 3 | Nail 0.5 mm thick | 2 | 0.3 |
| | Average across all nails | 25 | 2.8 |
| | Control (0.5 mm thick) | 390 | 39 |

The data show that:
Over a duration of 15 minutes the He/1%Ar plasma is able to act through varying thicknesses of human nail to reduce the cell count by around 95%,

Part C—Comparative Testing of Gas Mixes Using Medpharm Ltd Infected Nail Model Using Trichophyton Rubrum

The objective was to apply the successful gas mixes from part B to an industry recognised onychomycosis nail model to identify the most efficacious mix using the actual pathogen responsible for the majority of infections, and to optimise the mix and the application regime to maximise efficacy.

All of the following tests were carried out by Medpharm Ltd using their infected nail model (ChubTur®) whereby full thickness human nail samples are inoculated with spores of Trichophyton Rubrum and incubated for 14 days in a hydrated warm environment to allow the fungus to grow in to the nail. The nail is set in the ChubTur® cell apparatus and exposed to various regimes of plasma treatment using different gas mixes.

Measurements of effectiveness are derived from an ATP assay following 24 hrs incubation. In this model, the amount of luminescence measured is directly proportional to the amount of ATP present, where the level of ATP detected is an indication of the viability of T. Rubrum. Most experiments are based on a sample size of 6.

Test C1—Gas Mix Comparisons

Through numerous tests it was determined, that the maximum result measurable with the model was 95% kill of the organism. Therefore the time that various gas mixes took to achieve this level was assessed alongside the kill level achievable through a 6 minute application.

| Test | | Time to achieve 95% kill | Kill achieved in 6 |
|---|---|---|---|
| # | Gas used | (mins) | minutes (%) |
| 1 | He/1% Ar | 15 | 82% |
| 2 | H2/250 ppm O2 | 10 | 90% |
| 3 | Ar/4% Kr | 1.5 | 95% |

The data shows that:
A number of gas mixes can achieve 95% kill of the fungus through the nail given enough time.
The fastest result is achieved by the Ar/4%Kr mix which is 10× quicker than the He/1%Ar mix.

Test C2—Comparisons with Commercial Products

The aim of the study was to compare the in-vitro efficacy from a single 6 minute application of the various plasmas with single applications of commercial comparators as per the manufacturer's instructions—a topical anti-fungal cream and a cold laser device.

| Test | | |
|---|---|---|
| # | Description | Fungal kill achieved |
| 1 | He/1% Ar | 82% |
| 2 | H2/250 ppm O2 | 90% |
| 3 | Ar/4% Kr | 95% |
| 4 | Loceryl (topical anti-fungal by Galderma) | 10% |
| 5 | Non-thermal laser | 60% |

The data also shows that:
Single doses of plasma from a variety of gas mixes are significantly more effective than single doses of the commercial topical and cold laser comparators.
The most significant advantage over commercial comparators is achieved by the Ar/4%Kr mix.

Part D—Cosmetic Whitening of Teeth

The objective was to apply the successful gas mixes to demonstrate the potential for their use in the cosmetic whitening of teeth

Test D1—Stained HAP Disks

Hydroxyapatite disks are used as an enamel proxy for consistency and accessibility. Disks were etched with hydrochloric acid and then immersed in a tea/coffee solution for 4 days, rinsed, wiped and dried to leave only the stain that had penetrated in to the disk. The L*a*b* colour was measured using a spectrophotometer and then plasma was then applied to a masked off area of the disk for 5×2 minutes and the colour of this masked area remeasured following rehydration of the disk in distilled water to avoid recording temporary colour effects as a result of dehydration.

Gas flow rate was 2.5 SLPM; device voltage 7.5 kV; distance from exit tube to target 10 mm;

L*a*b* Colour Change of Stained HAP Disks Following 10 minutes of Plasma

| Gas mix | Mean delta L* | Mean delta a* | Mean delta b* |
|---|---|---|---|
| He | 2.40 | — | −0.70 |
| 1% Ar/He | 3.86 | −1.41 | −4.70 |
| 200 ppm O2/He | 3.73 | −1.45 | −4.97 |
| 500 ppm O2/He | 8.63 | −3.43 | −7.28 |
| 1% Ne/He | 2.76 | −1.33 | −3.20 |
| 4% Kr/Ar | 6.00 | −2.00 | −6.00 |

The data shows that:
Plasma can penetrate an enamel-like material and produce colour change in extrinsic and shallow intrinsic stains.
The gas blends produce greater colour change than He alone
The O2/He and Kr/Ar blends are the most effective.
L* (lightness) and b* (yellowness) dimensions both show significant improvement which are the most important to teeth colour.

Test D2—Human Enamel

Whole human teeth were cleaned and kept hydrated, L*a*b* colour measured using a spectrophotometer before being exposed to plasma treatment. No extra staining was applied. Colour was re-measured after at least 2 hours of rehydration in distilled water to avoid recording temporary colour effects as a result of dehydration.

Gas flow rate was 2.5 SLPM; device voltage 7.5 kV; distance from exit tube to target 10 mm; gas blend 1%Ar/He

Delta L* colour change of unstained human teeth following increasing rounds of 2 minute plasma treatments is shown in the table below.

| Delta L* | Round 1 | 2 | 3 | 4 | 5 | 6 |
|---|---|---|---|---|---|---|
| After | 1.50 | 2.33 | 3.02 | 3.93 | 4.12 | 3.77 |
| rehydration | | | | | | |

The data shows that:
Change in L* in human teeth is consistent with change in L* in stained HAP disks (after 10 minutes treatment using 1%Ar/He).
A sustainable colour change in tooth enamel is possible.
The maximum effect is achieved after 8-10 minutes.

Test D3—Enamel Penetration

Slices of human tooth enamel approximately 1 mm thick were stained front and back with melanin as an indicator of bleaching effect by plasma. The stained faces of the slices were colour measured as above, then placed on clean hydroxyapatite disks and the edges sealed to prevent leakage of plasma around the sides.

Multiple rounds of 2 minute plasma treatments were applied as above.

Delta L* colour change in melanin stained top and bottom enamel surfaces following rounds of 2 minute plasma treatment is shown in the table below.

| Delta L* | Round 2 | 4 | 6 | 8 | 10 |
|---|---|---|---|---|---|
| Top side | 17.01 | 20.70 | 21.05 | 21.03 | 22.48 |
| Under side | 4.32 | 6.17 | 6.27 | 9.79 | 11.43 |

The data shows that:
The gas blend based plasma can penetrate 1mm thick human tooth enamel and thus reach the dentin which carries most of a tooth's colour.
The top side is bleached quite quickly with most effect being seen after just 8 minutes.
The under-side effect is slower to build up and less than the top side effect but nevertheless still significant.

Test D4—Human Dentin Bleaching

Exposed dentin samples from sectioned human teeth were colour measured as above, plasma treated as above, rehydrated and re-measured.

Delta L* colour change in dentin following rounds of 2 minute plasma treatment is shown in the table below.

| Delta L* | Round 1 | 3 | 5 | 7 | 9 |
|---|---|---|---|---|---|
| After | 1.37 | 4.15 | 4.08 | 3.90 | 4.75 |
| hydration | | | | | |

The data shows that:
The gas blend based plasma can produce material colour change in dentin once it passes through the enamel.

### Further Examples

A plasma device rig was connected to two different gases and the gas concentration measured by way of two mass flow controllers operated via a computer, as described in the methodology below. The plasma plume was first measured for temperature, ozone and nitrous oxide emissions, and voltage deposition, before attempting to bleach a ParaSure plasma indicator strip. The device head was left at a distance of 10 mm from the strip and the L* a* b* colour was measured at given intervals to a maximum of 1 hour. Results were found for a number of inert gas mixes, in addition to some molecular gas and inert gas mixtures

Apparatus:
Plasma Device
Two Alicat Mass Flow Controllers MC-10SLPM-D
Alicat USB Bus BB9
Laptop with FlowVisionMX control software
Konica Minolta Spectrophotometer CM-2600d
Tektronix Oscilloscope TDS2024C
TIM USB Thermal Camera
Fluke Thermometer 52 K/J
2BTechnologies Ozone Monitor 106-L
EnviroTechnology Nitrous Oxide Chemiluminescence Monitor 200E
Ocean Optics UV-NIR Spectrometer HR4000CG
“Wet Human” Test Model
1) Select correct gases on mass flow controllers and using the FlowVisionMX control software, select the appropriate gas concentration.
2) Set up nitrous oxide monitor. Ensure pump is running to draw sample gas through the system and sample tube is a close to the plume as possible.
3) Set up ozone monitor to record 10 minute averages during plasma treatment. The ozone monitor sample tube should also be placed as close to the plume as possible.
4) Set up Plasma device with the selected gas mixture using a flow rate of 1.51/min. Let gas flush through the 20-30 mins.
5) Switch on power to produce plasma, with power set to DC voltage 9.00 kV. Then measure:
   a. Peak to peak voltage on the human test model
   b. RMS voltage on the human test model
   c. Frequency on the human test model
   d. Frequency of the handpiece
6) After 10 minutes, record the nitrous oxide and ozone average readings.
7) Set up thermal imaging camera to find the temperature of the plume at the human test model. Allow sufficient time for the reading to stabilise before recording.
8) Record the temperature at the human test model using the thermometer, ensuring that the thermocouple is not directly in the plume.
9) Align optical fibre to ensure maximum readings for spectral data and record the spectrum with the electric dark spectrum over 1 second. Save the spectrum in .spc format for later analysis,
10) Repeat 5-9 at 7.00 kV and 5.00 kV.
11) Repeat 1 -10 for all necessary gas concentrations.
12) After completing the measurement matrix for each gas mixture, select the best two or three gas concentrations for bleach testing.
13) Calibrate the spectrometer and record the calibration data.
14) Mark a 3 mm target area on the plasma indicator.
15) Measure L*a*b* of the target using the spectrometer. Take 3 measurements for each sample, rotating sample by 90 degrees each time, Quote the average of these readings.
16) At 15 minutes, 30 minutes and 60 minutes, repeat 15.

Colour measurements using a spectrometer to observe L*a*b* values are well known in the art.

The spectral emissions for each gas concentration were measured to indicate which chemical species were being excited by the plasma plume at each voltage. It was discovered that the main bleaching agent in these tests was singlet Oxygen, although there is a notable bleaching effect that can be attributed to hydroxyl radicals.

In the Helium with added Argon gas mixtures, it was seen that the proportion of metastable Argon is strongly related to the amount of excited singlet Oxygen but less to the number of hydroxyl radicals. The highest levels of metastable Argon were found at 9.00 kV. There was found to be little relationship between the bleaching agents and metastable Helium.

A similar relation was found in the Helium/Neon gas mixtures; it was seen that the proportion of metastable Neon is linked to the amount of excited singlet Oxygen and hydroxyl radicals. The highest levels of metastable Neon were again found at 9.00 kV. This trend was not continued in the Argon/Krypton gas mix, as high levels of metastable Krypton led to varying levels of the bleaching agents. There was also a similar correlation with the bleaching gases in this mix and metastable Argon.

Helium/Xenon also did not fit the general trend, as the Xenon metastable was disproportionately high and did not seem to excite any other products. The limited results gained from Nitrogen gas mixes suggest that they also follow an alternative trend, however due to significant quenching giving such a small sample of data, the true method of bleaching remains unclear. The results from Hydrogen gas mixes show that hydroxyl radicals are the main bleaching agent in the plume. However this is likely due to the increased levels of hydrogen in the plasma itself.

The effectiveness of bleaching test varied considerably across the different gas mixtures and compositions. In general, the most efficient inert gas mixture was Argon/Krypton. However, it was seen that one particular composition of Helium/Neon was more effective. The least effective gas mixture was Helium/Argon, which was less than a tenth as effective as the Argon/Krypton mix.

Of the molecular gas mixtures, the most effective was Argon/Hydrogen. The molecular gases performed much worse when partnered with Helium. The Argon/Hydrogen mixtures are much more bleaching than the Helium/Hydrogen mix.

All percentages and ratios recited herein are by volume, unless otherwise stated.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. A plasma-generation device for applying plasma to a human body, the device comprising:
a reservoir containing a gas,
a plasma zone in fluid connection with the reservoir, and
means for generating a plasma by electrical discharge in the plasma zone,
wherein:
the gas comprises from 92% to 99.9% Argon and from 0.1% to 8% Krypton; or
the gas comprises from 95% to 99.5% Argon and from 0.5% to 5% Hydrogen: or
the gas comprises from 92% to 99.5% Argon and from 0.5% to 8% Nitrous
Oxide,

2. The plasma-generation device of claim 1, wherein:
the gas comprises from 94% to 99.5% Argon and from 0.5% to 6% Krypton; or
the gas comprises from 97.5% to 99% Argon and from 1% to 2.5% Hydrogen; or
the gas comprises from 94% to 98% Argon and from 2% to 6% Nitrous Oxide.

3. The plasma-generation device of claim 1, wherein the means for generating a plasma comprises a power supply and a dielectric electrode for placing in proximity to a human body, and wherein, in use, the plasma zone is formed between the dielectric electrode and a surface of a human body.

4. The plasma-generation device of claim 1, wherein the means for generating a plasma comprises a power supply, and first and second electrodes, and wherein, in use, the plasma zone is formed between the first and second electrodes and wherein a flow of gas from the reservoir through the plasma zone provides a flow of plasma to contact a surface of a human body.

5. The plasma-generation device of claim 1, wherein the means for generating a plasma comprises a power supply, and first and second electrodes sandwiching a dielectric material, and wherein, in use, the plasma zone is formed between the first or second electrode and a surface of a human body.

6. The plasma-generation device of claim 1, wherein the device is hand-held.

7. The plasma-generation device of claim 3, wherein the power supply comprises a battery integrated into a hand-held device.

8. The plasma-generation device of claim 1, wherein the gas is supplied through the means for generating a plasma at a flow rate of less than 5 l/min.

9. The plasma-generation device of claim 1, wherein the means for generating a plasma operates at a voltage of from 2-15 kV.

10. The plasma-generation device of claim 1, wherein the device is a hair straightener, a toothbrush, foot-spa or a hair-brash.

11. A refillable canister for use in a the plasma-generation device, the device comprising a reservoir containing a gas, a plasma zone in Quid connection with the reservoir, and means for generating a plasma by electrical discharge in the plasma zone, the canister comprising a reservoir and containing a pressurised gas, wherein:
the gas comprises from 92% to 99.9% Argon and from 0.1% to 8% Krypton; or
the gas comprises from 95% to 99.5% Argon and from 0.5% to 5% Hydrogen; or
the gas comprises from 92% to 99.5% Argon and from 0.5% to 8% Nitrous Oxide.

12. The refillable canister of claim 11, wherein the gas consists essentially of Argon and Krypton, Argon and Nitrous oxide, or Argon and Hydrogen, together with any unavoidable impurities,

13. The refillable canister according to claim 11, wherein the device is hand-held and wherein the canister is integrated into the hand-held device.

14. The use of a plasma for use in a treatment method, wherein the plasma is generated by electrical discharge through a gas, wherein
the gas comprises from 92% to 99.9% Argon and from 0.1% to 8% Krypton; or
the gas comprises from 95% to 99.5% Argon and from 0.5% to 5% Hydrogen; or
the gas comprises from 92% to 99.5% Argon and from 0.5% to 8% Nitrous Oxide.

15. The use of a plasma of claim 14, wherein the treatment method is for the cosmetic lightening of nails.

16. The use of a plasma of claim 14, wherein the treatment method is for the cosmetic whitening of teeth.

17. The use of a plasma of claim 16, wherein the treatment A method for the cosmetic whitening of a tooth comprises:
plasma treating a surface of a tooth.

18. The use of a plasma of claim 14, wherein the treatment method is for the cosmetic bleaching of hair.

19. The use of a plasma of claim 18, wherein the treatment method for the cosmetic bleaching of a hair comprises
plasma treating a surface of the hair.

20. The use of a plasma of claim 14, wherein the treatment is for the cosmetic dyeing of hair, the method comprising:
plasma treating a surface of the hair; and
applying a hair-dye to the plasma-treated hair.

21. A plasma generated by electrical discharge through a gas, wherein:
the gas comprises from 92% to 99.9% Argon and from 0.1% to 8% Krypton; or
the gas comprises from 95% to 99.5% Argon and from 0.5% to 5% Hydrogen; or
the gas comprises from 92% to 99.5% Argon and from 0.5% to 8% Nitrous Oxide.

22. The use of a plasma according to claim 14, wherein the plasma heats a surface to be treated to a temperature of 48° C. or lower.

23. (canceled)

24. The plasma-generation device of claim 8, wherein the gas flow rate is less than 2.5 l/min.

25. The plasma-generation device of claim 24, wherein the gas flow rate is less than 1.5 l/min.

26. The plasma generation device of claim 25, wherein the flow rate is from 0.1 to 0.5 l/min.

27. The use of a plasma of claim 14, wherein the treatment method is for treating a fungal infection in a nail.

28. The use of a plasma according to claim 22, wherein the temperature is 42° C. or lower.
